(19) United States
(12) Patent Application Publication
(10) Pub. No.: US 2010/0137579 A1
(43) Pub. Date: Jun. 3, 2010

(54) Process for purifying medical grade hyaluronic acid

(75) Inventors: Wei-Chih Huang, Tainan City (TW); Teh-Liang Chen, Tainan City (TW); Wen-Teng Wu, Tainan City (TW)

Correspondence Address: WPAT, PC;INTELLECTUAL PROPERTY ATTORNEYS, 2030 MAIN STREET, SUITE 1300, IRVINE, CA, 92614, US

(73) Assignee: Sustineo Biotechnology Co., Ltd., Tainan City (TW)

(21) Appl. No.: 12/325,830

(22) Filed: Dec. 1, 2008

(51) Int. Cl. C07H 1/06

(52) U.S. Cl. 536/123.1

(57) ABSTRACT

A process for purifying medical grade hyaluronic acid from a biological source under continual changed pH value is disclosed.

### FIELD OF THE INVENTION

The present invention relates to a process for purifying medical grade hyaluronic acid.

### BACKGROUND OF THE INVENTION

Hyaluronic acid (also called hyaluronan or hyaluronate, also known as HA) is a non-sulfated glycosaminoglycan and it is constituted by alternating and repeating units of D-glucuronic acid and N-acetyl-D-glucosamine, to form a linear chain having a molecular weight up to 13×10⁶ Daltons. It is known that hyaluronic acid distributed widely throughout connective, epithelial, and neural tissues. It is one of the chief components of the extracellular matrix, contributes significantly to cell proliferation and migration.

HA is highly hydrophilic, and in aqueous solutions shows high viscoelastic behavior and water binding capacity due to the high molecular weight and the high number of charged groups. Due to its properties, hydraluronic acid is able to retain the tonicity and elasticity of the skin. Accordingly, the application of HA will be easily found in cosmetics such as skin creams, cleansers, and facial aesthetics. Recent biomedical applications of HA include ophthalmic surgery, arthritis treatment, wound healing, embryonic development, tissue regeneration, tumorigenesis, etc.

Traditionally HA was extracted from animal tissue, such as rooster combs, pigskin, and fishskin, but nowadays is produced by the fermentation of bacteria. The fermentation of bacteria is more appropriate for mass production and easier to control the quality. The advantage of using fermentation of bacteria also includes cost-saving, high yield and allergy and virus particles avoidance. The most commonly used bacterial strain for the production of HA belongs to Streptococcus.

Several previous separation procedures have used multiple solvent precipitations, cationic detergent treatment, diafiltration, anion exchange resin treatment, protease digestion for purification of HA.

U.S. Pat. No. 4,517,295, U.S. Pat. No. 4,780,414, U.S. Pat. No. 5,563,051 and U.S. Pub. No. 20060127987 disclosed a process for producing HA by repeatedly using large amounts of organic solvent for precipitation. U.S. Pat. No. 6,489,467 disclosed a process for purifying high molecular weight hyaluronic acid from a biological source by adjusting the pH value and diafiltering. Haidong Zhou et al. teaches a two-stage tangential flow filtration process for the separation of hyaluronic acid from fermentation broth (Haidong Zhou et al. Separation and Purification Technology 52 (2006) 29-38); however, the membrane filtration system H. Zhou taught can not be mass production and the HA produced by the citied method can not reach the medical grade. These problems still have to be solved to meet the need of market.

### SUMMARY OF THE INVENTION

The present invention provides a process for purifying medical grade hyaluronic acid from a biological source comprises: a) centrifugating a fermentation broth and retaining a supernatant; b) adjusting pH value of the supernatant liquid to acidity and then microfiltrating the liquid; c) adjusting pH value of a filtrate of step b) to neutral on a reservoir; d) diafiltrating the filtrate by acidic solution and ultrafiltrating the filtrate on the reservoir under a through membrane pressure until the filtrate becoming acidity; e) adjusting diafiltrate to basicity; f) diafiltrating and ultrafiltrating under the through membrane pressure the filtrate on the reservoir until the filtrate becoming neutral; and g) diafiltrating the HA solution with reverse osmosis (RO) water.

### BRIEF DESCRIPTION OF DRAWINGS

FIG. 1 is the devise of HA purification.

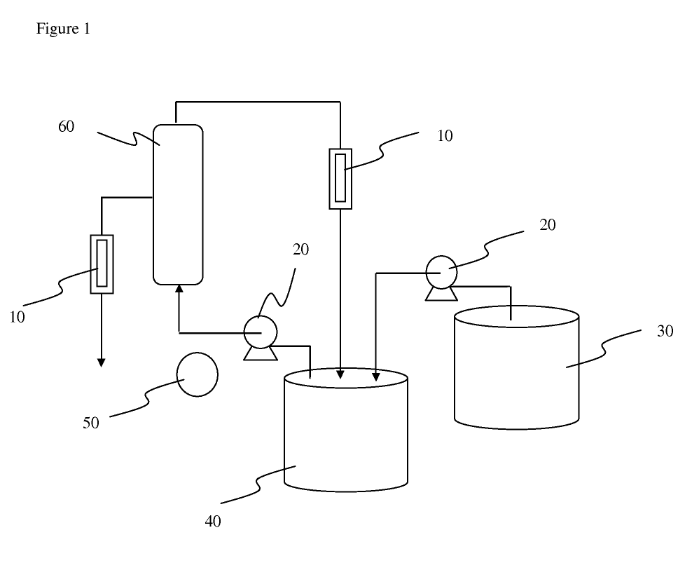

FIG. 2 shows the pH of broth effect the residual protein.

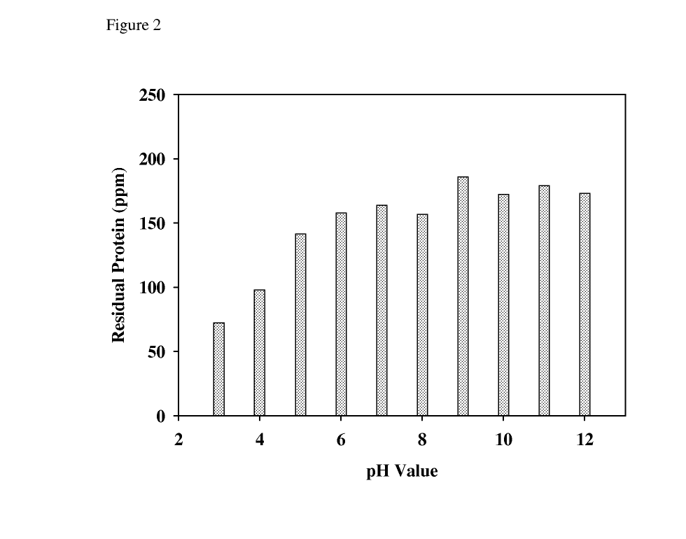

FIG. 3 shows the pH of HA continuously changed by the diafiltrate is helpful to reach the medical grade when the diafiltration was progressed.

### DETAILED DESCRIPTION OF THE INVENTION

In the meaning of the present invention, hyaluronic acid for medical use is defined as British Pharmacopoeia (2003). The appearance of solution should be clear and the readout number of absorbance no more than 0.01 at 600 nm, and the nucleic acid of solution should be less than 0.5 at 260 nm. The pH value of solution is between from 5.0 to 8.5. The ratio by weight of protein is under 0.1% and the ratio of chlorides is under 0.5%.

The present invention provides a process for purifying medical grade hyaluronic acid from a biological source comprises: a) centrifugating a fermentation broth and retaining a supernatant; b) adjusting pH value of the supernatant liquid to acidity and then microfiltrating the liquid; c) adjusting pH value of a filtrate of step b) to neutral on a reservoir; d) diafiltrating the filtrate by acidic solution and ultrafiltrating the filtrate on the reservoir under a through membrane pressure until the filtrate becoming acidity; e) adjusting diafiltrate to basicity; f) diafiltrating and ultrafiltrating under the through membrane pressure the filtrate on the reservoir until the filtrate becoming neutral; and g) diafiltrating the HA solution with reverse osmosis (RO) water;

In the embodiment of the present invention, the process for purifying medical grade hyaluronic acid from a biological source comprises: a) centrifugating a fermentation broth to remove cells and retaining a supernatant; b) adjusting pH value of the supernatant liquid to acidity and then microfiltrating the liquid; c) adjusting pH value of a filtrate of step b) to 7.0 on a reservoir; d) diafiltrating the filtrate by pH 3.5, 1.5 M NaCl and ultrafiltrating the filtrate on the reservoir under a through membrane pressure until the pH value of filtrate reaching 4.0; e) adjusting pH value of diafiltrate to 8.0; f) diafiltrating and ultrafiltrating the filtrate under the through membrane pressure on the reservoir until the pH value of filtrate reaching 7.0; and g) diafiltrating the supernatant liquid with RO water. The biological source set forth is Streptococcus species. In the embodiment of the present invention, the Streptococcus species is Streptococcus zooepidemicus.

The characterized in step b), the pH of supernatant liquid is around 3.0, and the pore size of microfiltration membrane is 0.1 to 1 μm. In the embodiment, the pore size of microfiltration membrane is 0.45 μm. The through membrane pressure set forth is between 0.1˜100 psi. In the embodiment of the present invention, the through membrane pressure is between 1˜50 psi. In the further embodiment, the through membrane pressure is 15 psi. The filtrates on the reservoir in steps d) and f) are constant in volume. Besides, the volume of filtrate is maintained by controlling flow rate of diafiltrate and permeant to be constant.

### EXAMPLE

### Example 1

### Fermentation

Cells of Streptococcus zooepidemicus (supplied by Food industry research and development institute) were cultivated in an appropriate medium as table 1. The fermenting tank (model MDL 300-3L, B.E.MARUBISHI Co.Ltd., Japan) was pre-set at a temperature 37° C., pH 7.0±0.2, and 400 rpm. The medium was sterilized and inoculated with 5% Streptococcus zooepidemicus culture. The broth was harvested until the concentration is close to 1.0 g/L.

TABLE 1
The composition of medium

| | |
|---|---|
| Glucose* | 20 g/L |
| Yeast Extract | 10 g/L |
| NaCl | 2.0 g/L |
| K₂HPO₄* | 2.5 g/L |
| MgSO₄•7H₂O* | 1.5 g/L |
| Trypton | 1.7 g/L |
| Soyton | 0.3 g/L |

*The signed ingredients should be sterilized separately with other ingredients to avoid reacting in the high temperature.

### Example 2

### HA Purification

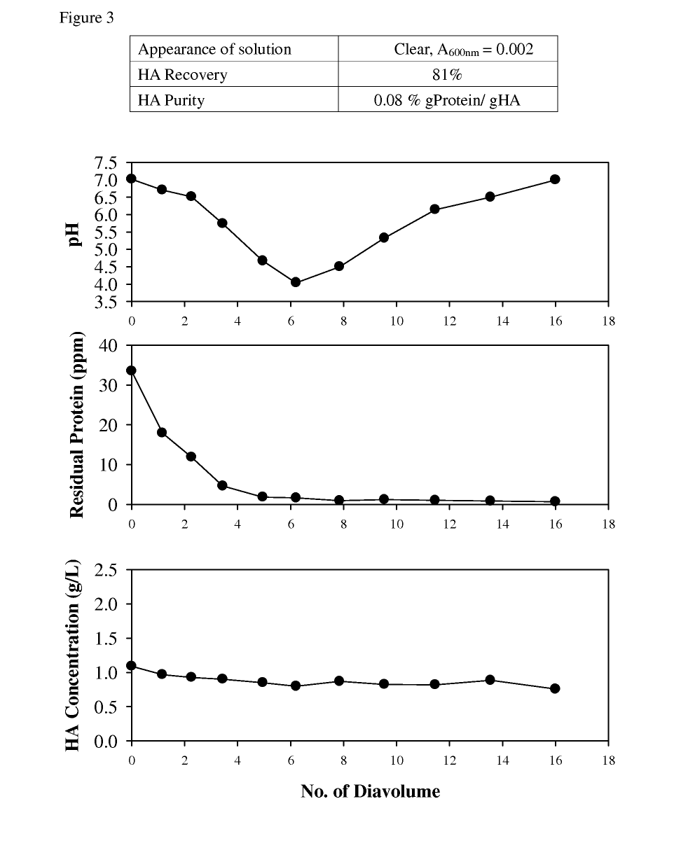

The broth as prepared above was centrifuged to remove the cells of Streptococcus zooepidemicus and retain the supernatant. First, the pH of supernatant was adjusted to 3.0 with HCl (5 N), thus obtaining a dreggy solution which contained roughly 50% protein aggregation. The solution was filtrated by a filter paper with pore size of 0.45 μm to remove the aggregate protein. The pH of filtrate was adjusted to 7 with NaOH (5 N), and then the solution was transferred into a reservoir. The solution was pumped into Ultrafiltration membrane whose retentate end was connected back to the reservoir by controlling the through membrane pressure at 15 psi. At the same time, the diafiltrate, pH 3.5, 0.15M NaCl, was added into the reservoir at the same flow rate of permeate end to maintain the concentration and reduce the pH of the HA in the reservoir. The pH of diafiltrate was changed to 8.0 until the pH of HA was reduced to 4.0. After 12 to 14 diafiltration volume, the pH of HA would be increase to about 7.0 and the HA solution was reach the medical grade. Finally, it required to change the diafiltrate to RO water to remove NaCl from the HA solution. The diafiltration with 4 diavolume RO water should be sufficient. The yield in HA had the following characteristics as FIG. 3.

TABLE 2
RO water was used to diafiltrate the HA solution in different pH.

| Diafiltrate | RO water | | | | | |
|---|---|---|---|---|---|---|
| pH | 6→4→4.7 | 5 | 6 | 7 | 8 | 9 |
| Aggregation | − | − | + | + | − | − |
| HA Recovery (%) | 88 | 71.9 | 97.0 | 98.0 | 72.2 | 44.2 |
| g Protein/g HA (%) | 0.37 | 0.14 | 0.24 | 0.63 | 0.23 | 0.26 |

TABLE 3
0.15 M NaCl was used to diafiltrate the
HA solution in different pH.

| Diafiltrate | 0.15 M NaCl | | | | |
|---|---|---|---|---|---|
| pH | 7→4→7 | 6→4→6 | 5 | 6 | 7 |
| Aggregation | − | − | − | + | + |
| g Protein/g HA(%) | 0.08* | 0.19 | 0.15 | | |

### Example 3

### Analytical method

Carbazole method was used to assay to the content of D-glucuronic acid in all the sample (Bitter T., et al., Anal. Biochem. 4 (1962) 330-334). Biorad Protein assay was used to decide the concentration of protein in the sample. The opacity density monitored under OD 600 nm was used to determine whether the protein aggregated or not. The quality of obtained HA complied with the specifications of British Pharmacopeia for medical grade HA (Table 4).

TABLE 4
properties of the HA from a typical batch

| Test | BP specifications* | Sample |
|---|---|---|
| Appearance of solution | Clear; A_{600 nm} ≦ 0.01 | Clear, A_{600 nm} = 0.002 |
| Ph | 5.0-8.5 | 7 |
| Nucleic acids | A_{260 nm} ≦ 0.5 | A_{260 nm} = 0.038 |
| Protein | ≦0.1% | 0.08% gProtein/gHA |
| Chlorides | ≦0.5% | 0.02% |
| Molecular Weight | None | 0.80 |
| (10⁶ Da) | | |
| % Na-Hyaluronic acid | Not less than 95% of dry | ≧98% |
| | material | |

*British pharmacopeia 2003

### DESCRIPTION OF MAJOR PARTS IN THE PRESENT INVENTION

10 Flow meter

20 Pump

30 Diafiltrate

40 Reservoir

50 Pressure meter

60 Ultrafiltration Membrane

1. A process for purifying medical grade hyaluronic acid from a biological source comprises:
a) centrifugating a fermentation broth and retaining a supernatant;
b) adjusting pH value of the supernatant liquid to acidity and then microfiltrating the liquid;
c) adjusting pH value of a filtrate of step c) to neutral on a reservoir;
d) diafiltrating the filtrate by acidic solution and ultrafiltrating the filtrate on the reservoir under a through membrane pressure until the filtrate becoming acidity;
e) adjusting diafiltrate to basicity;
f) diafiltrating and ultrafiltrating under the through membrane pressure the filtrate on the reservoir until the filtrate becoming neutral; and
g) diafiltrating the HA solution with water.

2. A process for purifying medical grade hyaluronic acid from a biological source comprises:
a) centrifugating a fermentation broth and retaining a supernatant;
b) adjusting pH value of the supernatant liquid to acidity and then microfiltrating the liquid;
c) adjusting pH value of a filtrate of step c) to 7.0 on a reservoir;
d) diafiltrating the filtrate by pH 3.5, 1.5 M NaCl and ultrafiltrating the filtrate on the reservoir under a through membrane pressure until the pH value of filtrate reaching 4.0;
e) adjusting pH value of diafiltrate to 8.0;
f) diafiltrating and ultrafiltrating the filtrate under the through membrane pressure on the reservoir until the pH value of filtrate reaching 7.0; and
g) diafiltrating the HA solution with water.

3. The process of claim 2, wherein the biological source is Streptococcus species.

4. The process of claim 3, wherein the Streptococcus species is Streptococcus zooepidemicus.

5. The process of claim 2, wherein the pH value of the supernatant liquid in step b) is 3.0.

6. The process of claim 2, wherein the supernatant liquid in step b) microfiltrates by a microfiltration membrane whose pore size is between 0.01 to 1 μm.

7. The process of claim 6, wherein the microfiltration membrane whose pore size is 0.45 μm.

8. The process of claim 2, wherein the through membrane pressure is between 0.1˜100 psi.

9. The process of claim 8, wherein the through membrane pressure is between 1˜50 psi.

10. The process of claim 9, wherein the through membrane pressure is 15 psi.

11. The process of claim 2, wherein the filtrates on the reservoir in steps d) and f) are constant in volume.

12. The process of claim 11, wherein the volume of filtrate is maintained by controlling flow rate of diafiltrate and permeate to be constant.
